(19) United States
(12) Patent Application Publication
(10) Pub. No.: US 2022/0126086 A1
(43) Pub. Date: Apr. 28, 2022

(54) METHOD AND SYSTEM FOR DETERMINING A FLOW SPEED OF A FLUID FLOWING THROUGH AN IMPLANTED, VASCULAR ASSISTANCE SYSTEM

(71) Applicant: KARDION GMBH, Stuttgart (DE)

(72) Inventors: Thomas Alexander Schlebusch, Renningen (DE); Tobias Schmid, Stuttgart (DE)

(21) Appl. No.: 17/274,354

(22) PCT Filed: Sep. 24, 2019
(86) PCT No.: PCT/EP2019/075662

§ 371 Date: Jan. 12, 2022

(30) Foreign Application Priority Data
Sep. 25, 2018 (DE) 10 2018 216 305.7

(51) Int. Cl. A61M 60/523; A61M 60/178; A61M 60/216

(52) U.S. Cl. CPC A61M 60/523; A61M 2205/3375; A61M 60/216; A61M 60/178

(57) ABSTRACT

The invention relates to a method for determining at least one flow parameter of a fluid (31) flowing through an implanted vascular support system (10), comprising the following steps: a) estimating the flow velocity of the fluid (31), b) carrying out a pulsed Doppler measurement using an ultrasound sensor (18) of the support system (10) in an observation window (201) inside the support system (10), wherein the observation window (201) is displaced at an observation window velocity which is determined using the flow velocity estimated in Step a), c) determining the at least one flow parameter of the fluid using at least one measurement result of the pulsed Doppler measurement or a measurement result of the pulsed Doppler measurement and the observation window velocity.

The invention relates to a method and a system for determining a flow velocity of a fluid flowing through an implanted vascular support system. The invention also relates to an implantable vascular support system for carrying out said method. The invention can in particular be used in (fully) implanted left ventricular assist devices (LVAD).

Integrating ultrasonic volume flow sensors in cardiac support systems in order to use them to determine the so-called pump volume flow, which quantifies the fluid volume flow through the support system itself, is well-known. The ultrasonic volume flow sensors can carry out pulsed Doppler measurements and/or use the pulsed wave Doppler (PWD) method. Said method requires only one ultrasound transducer element and allows precise selection of the distance of the observation window from the ultrasound element. In the known PWD systems, ultrasound pulses are emitted at a defined pulse repetition rate (PRF). The pulse repetition rate has to exceed twice the maximum occurring Doppler frequency shift so as to not violate the Nyquist theorem. If this condition is not met, aliasing occurs, i.e. ambiguities in the recorded frequency spectrum.

Due to the geometric design of the measurement setup in cardiac support systems (VAD), the measurement range or the observation window may be so far away from the ultrasound transducer that the signal transit time of the ultrasound pulse from the transducer to the measurement region and back to the transducer cannot be neglected. Since, when using the PWD method, a new ultrasound pulse may or should (at least theoretically) only be emitted when the preceding pulse no longer provides significant echoes, the signal transit time limits the maximum possible pulse repetition rate. Due to the usually high flow velocities prevailing in cardiac support systems and the geometric boundary conditions for the distance of the observation window from the ultrasound element, there is typically a violation of the Nyquist sampling theorem, which leads to ambiguities (aliasing) in the spectrum.

Cardiac support systems with ultrasound sensors that do not use the PWD method are usually equipped with two ultrasound transducers so that, even though the described transit time problem can occur, it can be solved in other ways if implemented appropriately. However, cardiac support systems with ultrasound sensors that use the PWD method are susceptible to the described effect, in particular in the case of moderate to high flow velocities. The requirement to select the defined pulse repetition rate in such a way that aliasing does not occur is currently the state of the art.

The object of the invention is to specify a method and provide a system, by means of which at least one flow parameter of a fluid flowing through an implanted vascular support system, in particular a flow parameter of blood flowing through an implanted vascular support system, can be determined reliably and precisely.

This object is achieved by the method specified in claim 1 and the system specified in claim 10 and claim 11. Advantageous embodiments of the invention are specified in the dependent claims.

Proposed here according to claim 1 is a method for determining at least one flow parameter, in particular a flow velocity of a fluid flowing through an implanted vascular support system, comprising the following steps:
a) estimating the flow velocity of the fluid,
b) carrying out a pulsed Doppler measurement using an ultrasound sensor of the support system in an observation window inside the support system, wherein the observation window is displaced at an observation window velocity which is determined using the flow velocity estimated in Step a),
c) determining the at least one flow parameter of the fluid using at least one measurement result of the pulsed Doppler measurement or a measurement result of the pulsed Doppler measurement and the observation window velocity.

The vascular support system is preferably a cardial support system (or cardiac support system), particularly preferably a ventricular support system. The support system is usually used to support the conveyance of blood in the circulatory system of humans, e.g. a patient. The support system can be disposed at least partially in a blood vessel. The blood vessel is the aorta, for example, in particular in the case of a left ventricular assist device, or the common trunk (truncus pulmonalis) into the two pulmonary arteries, in particular in the case of a right ventricular assist device. The support system is preferably disposed at the outlet of the left ventricle of the heart or the left ventricle. The support system is particularly preferably disposed in aortic valve position.

The method can contribute to the determination of a fluid flow velocity and/or a fluid volume flow from a ventricle of a heart, in particular from a (left) ventricle of a heart toward the aorta in the region of a (fully) implanted, (left) ventricular (cardiac) support system. Said fluid is usually blood. The flow velocity is usually a primary velocity component of a fluid flow, in particular blood flow. The flow velocity is determined in a fluid flow or fluid volume flow which flows through the support system, in particular through a channel of the support system. The method advantageously allows the flow velocity and/or the fluid volume flow of the blood flow to be determined with high quality even outside the surgery scenario, in particular by the implanted support system itself.

The solution proposed here contributes in particular to the compensation of aliasing effects (or “spectrum wrapping”) in a medical pulsed-wave Doppler system. The method presented here can furthermore also advantageously contribute to a reduction of the spectral widening of the peak in the Doppler spectrum, which represents the various blood flow velocities occurring in the blood flow and transformed into the frequency domain. The method in particular serves to eliminate aliasing effects, reduce the spectral width of the signal from moving scatterers and/or widen the signal from static scatterers when measuring a fluid flowing through an implanted vascular support system.

A central aspect of the solution presented here can in particular also be seen in displacing the observation window of the PWD measurement setup at the approximate flow velocity of the blood, so that the resulting velocity difference between the blood and the displacement velocity of the observation window can be displayed in a range that can be measured unambiguously at the selected pulse repetition rate (PRF). The Doppler frequency resulting from this velocity difference can advantageously be determined without ambiguity.

This helps in particular to achieve the following particularly advantageous effects (at least to a significant extent):
Aliasing is eliminated, in particular by creating an unambiguity of the Doppler frequency.
Frequency peaks in the Doppler spectrum caused by moving scatterers are narrowed. This increases their amplitude and they are (better) visible against the background noise. This also increases the resolution of the measuring system.
Frequency peaks in the Doppler spectrum caused by static scatterers are narrowed. This reduces their amplitude and the signal energy is smeared in the spectrum.

In addition to the elimination of aliasing effects, an effect to be emphasized in this context is in particular that Doppler signals from moving scatterers are narrowed. In other words, this can also be described as a reduction of the “spectral widening” (the primary frequency component) of the measured relative Doppler shift between the observation window velocity and the flow velocity. Displacing an observation window in the direction of flow in particular increases the dwell time of the scatterers in the observation window and thereby reduces the spectral width of the Doppler frequency components of the corresponding flow velocities in the spectrum.

The same effect in particular has the reverse effect on the static scatterers, e.g. in the surrounding tissue. The shorter dwell time in the observation window leads to a smearing of the energy in the spectrum, which reduces the interference potential of these reflections.

In Step a), the flow velocity of the fluid is estimated. In other words, this means in particular that in Step a) the flow velocity, which is determined (e.g. calculated) in Step c), is (roughly) estimated first. This estimation is advantageously based on a previously performed ultrasound measurement (e.g. with a fixed observation window) using the ultrasound sensor of the support system. However, this is only an example. The estimation could, for example, also be based on an empirical value, for example based on the age of the patient and/or the severity of the patient's disease. Corresponding empirical values could, for example, be stored in a table that can be accessed by a control device of the support system. It can also be provided that the flow velocity, in particular in the case of high levels of support, is estimated on the basis of the electronic performance of a flow machine of the support system.

In Step b), a pulsed Doppler measurement is carried out using an ultrasound sensor of the support system in an observation window inside the support system, wherein the observation window is displaced at an observation window velocity which is determined using the flow velocity estimated in Step a). The ultrasound sensor can be an ultrasound transducer, for example. Preferably, a single (only one) ultrasound sensor is provided. The ultrasound sensor furthermore preferably comprises a single (only one) ultrasound element, which is advantageously formed with a piezo element.

The observation window is advantageously (always) located in a channel of the support system (in particular one through which the flow is as uniform as possible). A primary emission direction of the ultrasound sensor typically passes through the channel of the support system and the observation window (through the center or centrally). The primary emission direction of the ultrasound sensor in particular points into the channel of the support system. The observation window preferably passes through this region (the channel) as radially centered as possible. The observation window is usually displaced at the observation window velocity along the primary emission direction of the ultrasound sensor and along the flow direction of the fluid. The observation window velocity is in particular determined as a function of the flow velocity estimated in Step a). The observation window is in particular displaced at the previously determined (estimated), approximate flow velocity of the fluid (blood).

In Step c) the at least one flow parameter of the fluid is determined using a measurement result of the pulsed Doppler measurement and/or a measurement result of the pulsed Doppler measurement and the observation window velocity. The flow parameter is preferably a flow velocity and/or a fluid volume flow. If the cross-section through which the fluid can flow (for example the cross-section of the channel) and the flow profile are known, the fluid volume flow can be determined directly from the flow velocity. Stored calibration data is used for this purpose as appropriate. This calibration data makes it possible to infer the average flow velocity from a central flow velocity, assuming the flow profile applicable to each central flow velocity, and then calculate the volume flow by multiplying said average flow velocity by the cross-section. If necessary, the contribution of the flow profile can also be calculated via a final correction of the volume flow.

Alternatively or cumulatively to the flow velocity and/or the fluid volume flow, the flow parameter can, for example, also be a (blood) viscosity and/or a hematocrit value. This is in particular intended to illustrate that the Doppler spectrum (which is improved according to the solution presented here) can also be further processed or used apart from the flow velocity measurement. For example, the Doppler spectrum (which is improved according to the solution presented here) can be used as an input data set for further signal processing methods for determining further vital and/or system parameters, for example for estimating the blood viscosity and/or the hematocrit value.

The measurement result of the pulsed Doppler measurement can be available as a peak, for example. For example, by means of a calibration, specific values of the flow parameter, such as specific values for the velocity, the viscosity and/or the hematocrit value, can be assigned to specific peaks in the spectrum. This allows an exemplary and particularly advantageous determination of the mentioned parameters by comparison with stored calibration data.

According to one advantageous configuration, it is proposed that in Step c) a flow velocity of the fluid be determined using a measurement result of the pulsed Doppler measurement and the observation window velocity. Advantageously, in Step c), the Doppler spectrum of the velocity difference between the flow velocity of the fluid (blood flow velocity) and the observation window velocity (displacement velocity of the observation window) is calculated. Further preferably, the primary frequency component of the Doppler spectrum of the velocity difference (e.g. simple frequency peak or template matching of the expected frequency distribution) is determined in Step c).

In particular, in Step b), a new ultrasound pulse is only emitted when an echo (from a desired observation window distance to the ultrasound element) of an ultrasound pulse emitted immediately prior has been received. A new ultrasound pulse is preferably only emitted when all (significant) echoes of an ultrasound pulse emitted immediately prior have been received.

According to one advantageous configuration, it is proposed that in Step a) the estimation be carried out on the basis of at least one operating parameter of a flow machine of the support system. In other words, this means in particular that in Step a) the flow velocity is advantageously estimated on the basis of or as a function of at least one operating parameter of a flow machine of the support system. The fact that, based on the motor characteristic map of the flow machine, a rough estimation of the pump flow is possible from (only) the rotation rate of the drive or on the basis of the differential pressure across the flow machine and the rotation rate can thereby be utilized in a particularly advantageous manner.

The operating parameter of the flow machine is preferably at least a rotational speed, a current, an output or a pressure. The operating parameter is preferably a rotational speed (or rotation rate) of the flow machine, for instance of a drive (e.g. an electric motor) and/or a paddle wheel of the flow machine. The approximate viscosity of the patient's blood is usually known. The at least one operating parameter furthermore preferably includes a rotational speed of the flow machine and a differential pressure across the flow machine. The operating parameter is preferably used to determine (estimate) an estimated flow velocity of the fluid. A characteristic map, for example, in which the estimated flow velocity is stored as a function of the at least one operating parameter can be used to do this.

Alternatively or cumulatively, an ultrasound measurement using the ultrasound sensor of the support system can be carried out in Step a). Said ultrasound measurement is preferably not a pulsed Doppler measurement. Rather, an ultrasound measurement using two ultrasound sensors can be carried out, for example, whereby said sensors could, for example, be provided for this purpose in the support system.

According to one advantageous configuration, it is proposed that the observation window velocity be determined such that a (relative) Doppler shift (between the flow velocity and the observation window velocity) is transformed into a range that can be displayed without ambiguity. In other words, it can also be said that the observation window is moved at a velocity that transforms the relative Doppler shift between the blood flow and the velocity of the observation window into a range that can be displayed without ambiguity, in particular with the selected ultrasound frequency and PRF.

The observation window velocity is preferably determined in such a way that there is a velocity difference or relative velocity between the (estimated or to be determined) flow velocity of the fluid and the observation window velocity that is displayed in a range that can be measured unambiguously, in particular with the selected pulse repetition rate (PRF). In this context, it is particularly preferably provided that a Doppler frequency resulting from this velocity difference or relative velocity can be determined without ambiguity.

According to one advantageous configuration, it is proposed that the observation window velocity correspond substantially to the flow velocity estimated in Step a). The term “substantially” here includes deviations of no more than 10%. This advantageously contributes to setting a velocity difference or relative velocity between the flow velocity of the fluid and the observation window velocity to be as low as possible, which generally has an advantageous effect on a display in the Doppler frequency spectrum that is as unambiguous as possible. It is particularly preferred if the observation window velocity corresponds to the flow velocity estimated in Step a).

According to one advantageous configuration, it is proposed that, to displace the observation window, the time interval between an emission of an ultrasound pulse and a (start time of a) measuring time interval from ultrasound pulse to ultrasound pulse be changed. In particular, to displace the observation window and/or to set the observation window velocity, the time interval between a time of emission of an ultrasound pulse and a start time of a measuring time interval from an ultrasound pulse to an (immediately) following ultrasound pulse is changed, in particular extended or shortened.

The position of the observation window (measurement region) can usually be specified or set via time intervals (if the speed of sound in the fluid is known). In an ultrasound measurement, reflections from scatterers (e.g. blood cells) are usually received directly in front of the transducer immediately (in terms of time) after the emission of an ultrasound pulse. Then, as the wavefront advances further, reflections from more distant regions are received.

In a pulsed Doppler method, the received signals are typically processed only in a specific time interval temporally spaced apart from the time of emission of the ultrasound pulse. In other words, this means in particular that the start time of a measuring time interval having a start time and an end time is temporally spaced apart from a time of emission of the ultrasound pulse and that only the reflections of the emitted ultrasound pulse received in this measuring time interval are evaluated.

The spatial distance of the observation window to the ultrasound sensor, in particular to the transducer plane of the ultrasound transducer, can be selected or set via the selection of the time interval between the time of emission and the start time of the measuring time interval. The spatial extent of the observation window along the main beam direction of the ultrasound transducer can be selected or set via the length of the time interval (time interval between the start time and the end time of the measuring time interval).

A pulsed Doppler measurement usually consists of a large number of individual ultrasound pulses, i.e. a rapid sequence of emission and reception times with the frequency PRF (pulse repetition frequency). In this context, the PRF is in particular the duration from emission pulse to emission pulse. Changing the time interval between the time of emission and the start time of the measuring time interval from pulse to pulse, results in a moving observation window.

In this context, the observation window velocity can, for example, be set as follows: If the start time of the emission of an ultrasound pulse occurs at the time point tₒ, the start time of the observation (in the receiver) occurs at the time n·t_{beo,start} (whereby n is a natural number) and the speed of sound c₀ in blood is known, then the distance sₙ between the ultrasound transducer and the start of the observation window is:

$${s}_{n} = {c}_{0} \cdot \frac{n \cdot {t}_{beo , start} - {t}_{0}}{2}$$

If said distance is now related to the “sampling rate” 1/PRF (the time that passes between two pulses), then the observation window moves away from the ultrasound transducer at the following velocity v_{beo,start}:

$${v}_{beo , start} - \frac{{s}_{n}}{1 / PRF} = {s}_{n} \cdot PRF$$

According to one advantageous configuration, it is proposed that the observation window velocity and a sampling rate (of the evaluation unit (measuring unit) or the control and/or processing device) be adapted to one another. This can advantageously contribute to improving the signal-to-noise ratio (SNR). In this context, the sampling rate contributes in particular to the evaluation of the received signal or the reflected and received ultrasound pulses. In this context, it is particularly advantageous if the observation window velocity and a sampling rate are adapted to one another according to the following equation:

$${v}_{Gate} = n \cdot \frac{PRF \cdot {c}_{0}}{2 \cdot {f}_{S}} n ∈ Z$$

vGate here describes the observation window velocity, n is any whole number, PRF is the pulse repetition rate, c0 is the speed of sound in the fluid and fS is the sampling rate.

According to one advantageous configuration, it is proposed that the observation window velocity be determined such that the measurement result of the pulsed Doppler measurement and a Doppler shift caused by static scatterers (in the spectrum) are spaced apart from one another. This advantageously makes it possible to prevent the sought Doppler frequency shift from being covered by the frequency peak in the Doppler spectrum caused by static scatterers, e.g. the aortic wall. The observation window velocity is preferably determined in such a way that the measurement result of the pulsed Doppler measurement and a Doppler shift caused by static scatterers are not displayed on top of one another in the spectrum and/or can be separated.

In the method described here (displacing the observation window), reflections caused by the non-moving scatterers, e.g. by the aortic wall, are no longer displayed, in particular not at a Doppler frequency of 0 Hz, but are instead shifted according to the velocity of the observation window with a resulting Doppler frequency. This can lead to undesired covering or superpositioning in the Doppler spectrum, which can advantageously be avoided by an in particular slight change in the observation window velocity.

According to one advantageous configuration, it is proposed that in Step c) a or the flow velocity of the fluid be determined by adding together the observation window velocity and a relative velocity determined on the basis of the pulsed Doppler measurement. In this context, the actual flow velocity of the fluid is preferably determined by adding together the known observation window velocity and the relative velocity determined by the measurement.

According to a further aspect, an implantable vascular support system configured to carry out a here proposed method is proposed. The support system preferably comprises an (electronic) control and/or processing device (measuring unit), which is configured to carry out a here proposed method.

The support system is preferably a left ventricular cardiac support system (LVAD) or a percutaneous, minimally invasive left ventricular assist device. The system is furthermore preferably fully implantable. In other words, this means in particular that the means required for determination, in particular the ultrasound sensor, are completely inside the body of the patient and remain there. The support system can also be designed in multiple parts or comprise a plurality of components that can be disposed spaced apart from one another, so that the ultrasound sensor and a control and/or processing device (measuring unit) of the support system that can be connected to said ultrasound sensor, for example, can be disposed separated from one another by a wire. In the multipart design, the control and/or processing device disposed separate from the ultrasound sensor can likewise be implanted or disposed outside the patient's body. Either way, it is not absolutely necessary for the control and/or processing electronics to also be disposed in the body of the patient. For example, the support system can be implanted such that the control and/or processing device is disposed on the patient's skin or outside the patient's body and a connection to the ultrasound sensor system disposed in the body is established. The support system is particularly preferably configured and/or suited to being disposed at least partially in a ventricle, preferably in the left ventricle of a heart, and/or in an aorta, in particular in aortic valve position.

The support system furthermore preferably comprises a channel, which is preferably formed in an (inlet) tube or an (inlet) cannula, and a flow machine, such as a pump and/or an electric motor. The electric motor is a routine component of the flow machine. The channel is preferably configured such that, in the implanted state, it can guide fluid from a (left) ventricle of a heart to the flow machine. The support system is preferably elongated and/or hose-like. The channel and the flow machine are preferably provided in the region of oppositely disposed ends of the support system.

In particular, exactly or only one ultrasound sensor is provided. The ultrasound sensor preferably comprises exactly or only one ultrasound transducer element. This is in particular sufficient for a Doppler measurement if the PWD method is used.

The system specified in claim 11, which comprises an implantable vascular support system and comprises a control and/or processing device for determining at least one flow parameter of a fluid flowing through the implantable vascular support system, includes:
a) a device for estimating the flow velocity of the fluid,
b) a device for carrying out a pulsed Doppler measurement using an ultrasound sensor in an observation window inside the support system, wherein the observation window is displaced at an observation window velocity which is determined using the flow velocity estimated in step a),
c) a device for determining the at least one flow parameter of the fluid using at least one measurement result of the pulsed Doppler measurement or a measurement result of the pulsed Doppler measurement and the observation window velocity.

The device for determining the at least one flow parameter of the fluid can be designed to determine a flow velocity of the fluid using a measurement result of the pulsed Doppler measurement and the observation window velocity.

The device for estimating the flow velocity of the fluid can in particular be designed to estimate the flow velocity of the fluid on the basis of an operating parameter of a flow machine of the support system.

It is advantageous if the observation window velocity of the observation window of the device for carrying out a pulsed Doppler measurement is designed to transform a Doppler shift into a range that can be displayed without ambiguity.

In particular, it is advantageous if the observation window velocity corresponds substantially to a flow velocity estimated in the device for estimating the flow velocity of the fluid.

The device for carrying out a pulsed Doppler measurement can in particular be designed to displace the observation window by changing the time interval between an emission of an ultrasound pulse and a measuring time interval from ultrasound pulse to ultrasound pulse.

The observation window velocity and a sampling rate of the fluid flowing through the implanted vascular support system can in particular be adapted to one another.

One advantageous embodiment of the system provides for the observation window velocity to be determined such that the measurement result of the pulsed Doppler measurement and a Doppler shift caused by static scatterers are spaced apart from one another.

It can in particular be provided that, in the system for determining at least one flow parameter of a fluid flowing through a vascular support system that can be implanted in the human body, the device for determining the at least one flow parameter of the fluid is designed to determine the flow velocity of the fluid by adding together the observation window velocity and a relative velocity determined on the basis of the pulsed Doppler measurement.

The details, features and advantageous configurations discussed in connection with the method can correspondingly also occur in the support system presented here and vice versa.

In this respect, reference is made in full to the statements there for a more detailed characterization of the features.

The solution presented here as well as its technical environment are explained in more detail below with reference to the figures. It is important to note that the invention is not intended to be limited by the design examples shown. In particular, unless explicitly stated otherwise, it is also possible to extract partial aspects of the facts explained in the figures and to combine them with other components and/or insights from other figures and/or the present description. The figures show schematically:

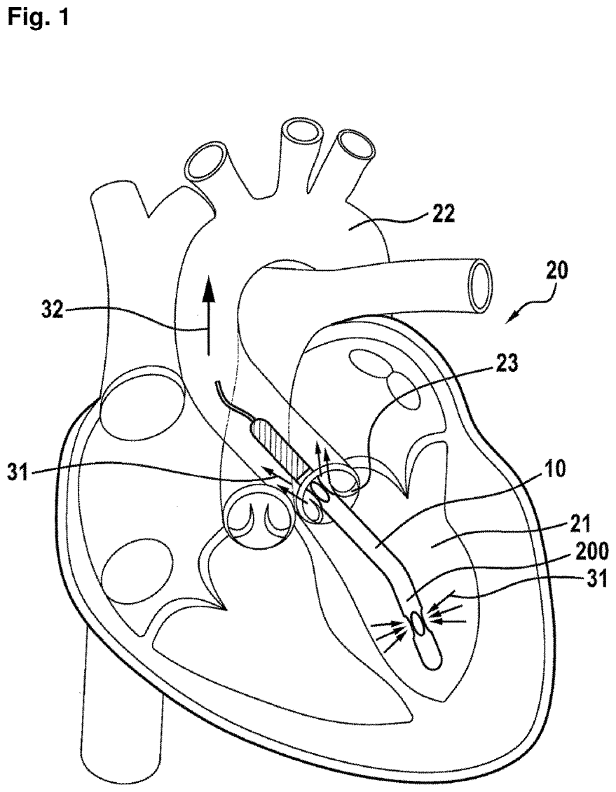

FIG. 1: an implanted vascular support system in a heart,

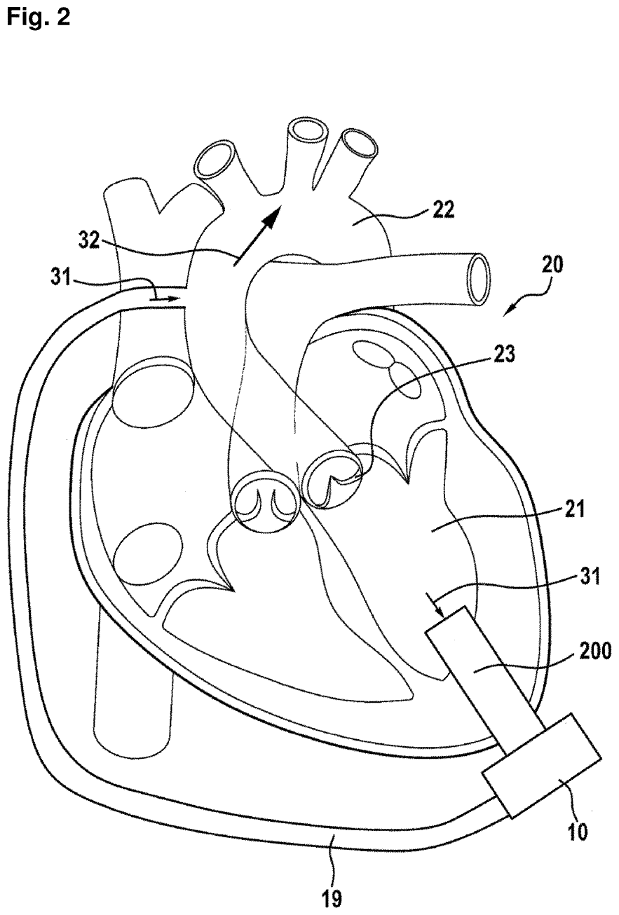

FIG. 2: a further implanted vascular support system in a heart,

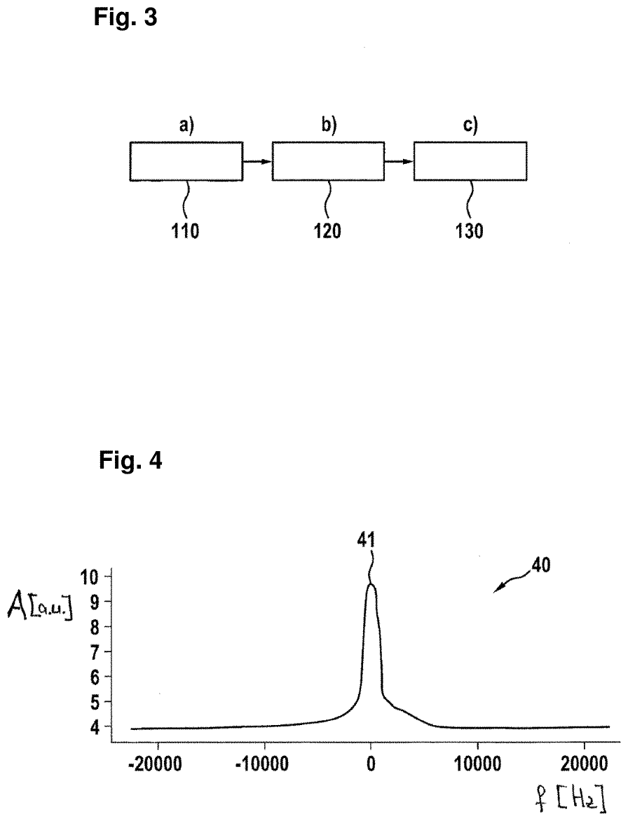

FIG. 3: a sequence of a here presented method,

FIG. 4: an example Doppler frequency spectrum,

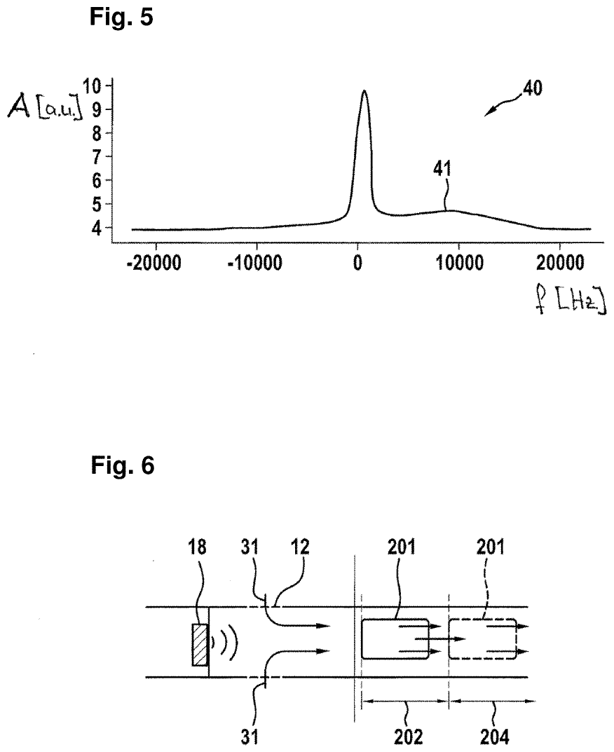

FIG. 5: a further example Doppler frequency spectrum,

FIG. 6: a detail view of a here proposed support system,

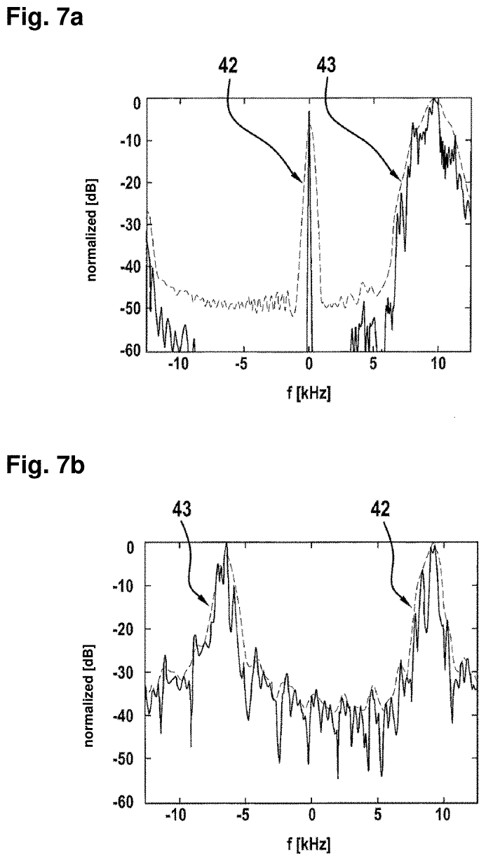

FIG. 7: example Doppler frequency spectra,

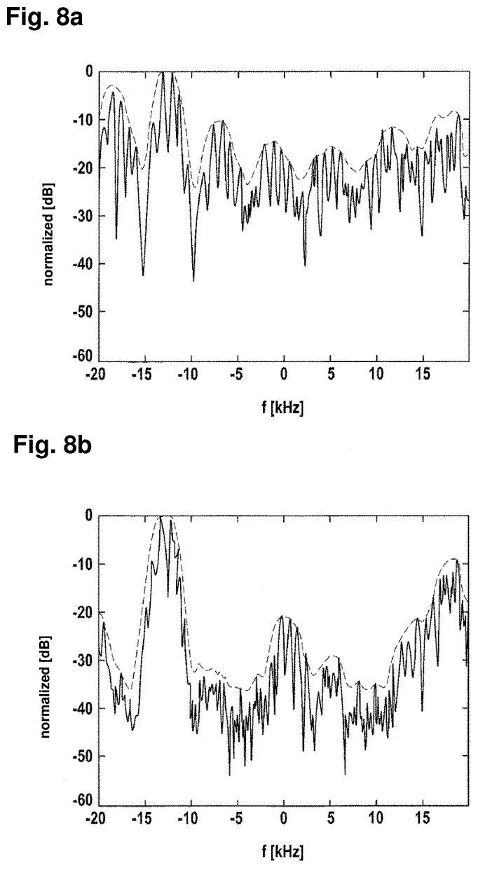

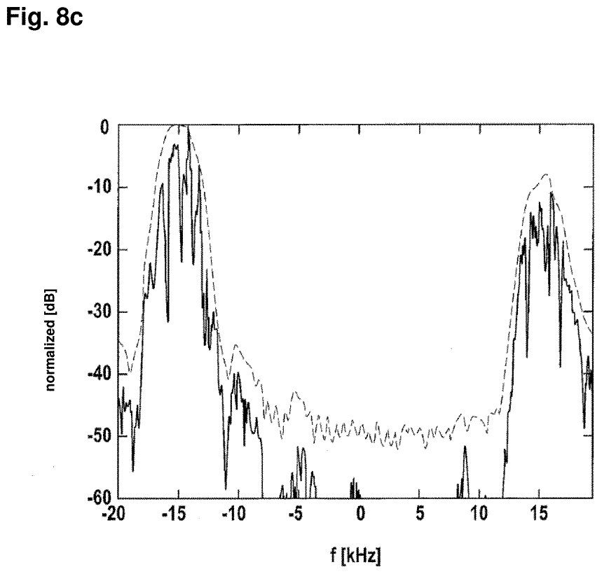

FIG. 8: further example Doppler frequency spectra,

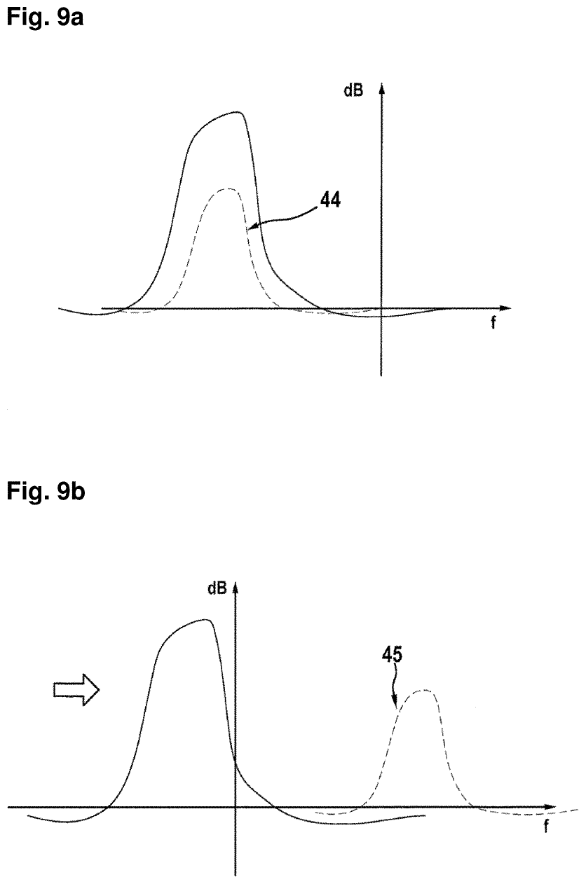

FIG. 9: further example Doppler frequency spectra, and

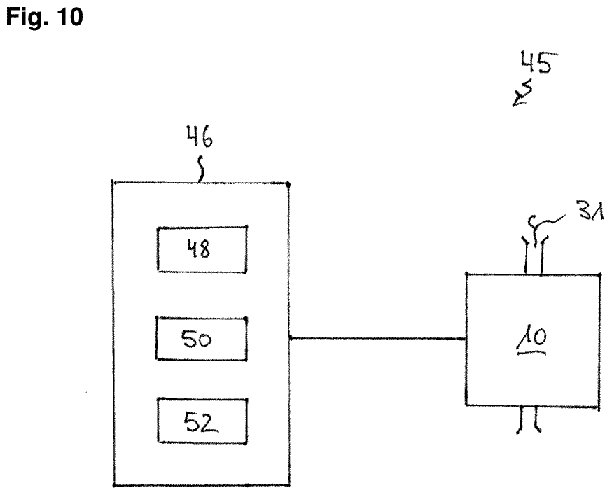

FIG. 10: a system comprising an implantable vascular support system and comprising a control and/or processing device for determining at least one flow parameter of a fluid flowing through the implantable vascular support system.

FIG. 1 schematically shows an implanted vascular support system 10 in a heart 20. The support system 10 supports the heart 20 by helping to convey blood from the (left) ventricle 21 into the aorta 22. For this purpose, the support system 10 is anchored in the aortic valve 23, as exemplified by FIG. 1. At a level of support of 100%, the support system 10 (LVAD) conveys the entire blood volume flow. The level of support describes the proportion of the volume flow conveyed through the support system 10 by a conveying means, such as a pump of the support system 10, to the total volume flow of blood from the ventricle 21 to the aorta 22.

Accordingly, at a level of support of 100%, the total fluid volume flow 32 from the ventricle 21 and the fluid volume flow 31 through the support system 10 are identical. The aortic valve or bypass volume flow (not shown here; symbol: Qₐ) is consequently zero. The total fluid volume flow 32 can also be described as (total) cardiac output (CO, symbol: Q_{CO}). The fluid volume flow 31 can also be referred to as a so-called pump volume flow (symbol: Qₚ), which quantifies only the flow through the support system 10 itself. The level of support can thus be calculated from the ratio Qp/Q_{CO}.

As an example, the support system 10 according to FIG. 1 is a cardiac support system in aortic valve position. The cardiac support system 10 is positioned in a heart 20. Blood is withdrawn from ventricle 21 and delivered into the aorta 22. The operation of the cardiac support system 10 (pump part) produces a blood flow 31.

In support systems 10 of the type shown in FIG. 1, the blood is conveyed inside a cannula-like section or channel 200 of the (cardiac) support system 10 through the aortic valve 23 and discharged again in the region of the aorta 22. The tip of the support system 10 (which projects into the ventricle 21) is particularly preferably suitable for the integration of an ultrasound transducer, so that the blood then flows away from the ultrasound transducer into the cannula-like section or channel 200 of the (cardiac) support system 10.

FIG. 2 schematically shows a further implanted vascular support system 10 in a heart 20. As an example, the support system 10 according to FIG. 2 is a cardiac support system in apical position. The reference signs are used consistently, so that reference can be made in full to the preceding statements regarding FIG. 1.

In support systems 10 of the type shown in FIG. 2, the blood is drawn in through a cannula-like section or channel 200 and returned to the aorta 22 through a bypass 19 outside the heart 20. In this case, the integration of an ultrasound transducer in the pump housing of the (cardiac) support system 10, looking out of the cannula-like section 200 drawing in the blood in the direction of the ventricle 21, is most suitable. In other words, this means in particular that the ultrasound transducer is disposed in the support system 10, and is oriented toward the channel 200 and toward the ventricle 21. In this case, the blood flows toward the ultrasound transducer. The method proposed here works equally well with both variants of FIG. 1 and FIG. 2, because only the movement direction of the measurement window has to be adjusted (for example in a computer program).

FIG. 3 schematically shows a sequence of a method presented here in a system for determining at least one flow parameter of a fluid flowing through an implantable vascular support system.

The method is used to determine a flow velocity of a fluid flowing through an implanted vascular support system. The shown sequence of the method steps a), b) and c) with Blocks 110, 120 and 130 is only an example and can be the result of a regular operating sequence. In Block 110, the flow velocity of the fluid is estimated. In Block 120, a pulsed Doppler measurement is carried out using an ultrasound sensor of the support system in an observation window inside the support system, whereby the observation window is displaced at an observation window velocity which is determined using the flow velocity estimated in Step a). In Block 130, the at least one flow parameter of the fluid is determined using at least one measurement result of the pulsed Doppler measurement and/or a measurement result of the pulsed Doppler measurement and the observation window velocity.

For an example illustration of the method, the following parameters are assumed:
Diameter inlet or measurement region, e.g. 5 mm,
Maximum blood flow to be measured, e.g. Q=9 l/min,
Resulting max. blood flow velocity: v_{Blood,max}=7.64 m/s,
Speed of sound in blood, e.g. c_{Blood}=1540 m/s,
Ultrasound frequency, e.g. f₀=6 MHz,
Distance of ultrasound element to the beginning of the viewing window, e.g. 25 mm,
Number of ultrasonic oscillation cycles per emitted ultrasound PWD pulse, e.g. 10,
Resulting length of the wave packet produced by the ultrasound pulse (in distance): I_{Burst}=c₀×10/f₀=2.57 mm,
Resulting maximum propagation distance of ultrasound pulse: d=55.13 mm.

For a measurement directly in the direction of emission (flow direction corresponds to the primary emission direction; α=0), these specifications result in the following (expected) maximum Doppler shift:

$$df = \frac{2 \cdot {v}_{Blood , max} \cdot {f}_{0}}{{c}_{0}} = \frac{2 \cdot 7 , 64 \frac{m}{s} \cdot 6 MHz}{1540 \frac{m}{s}} = 59 , 53 kHz \qquad ( 1 )$$

The measurement should be carried out as a pulsed Doppler measurement, in which a new ultrasound pulse is emitted only when all significant echoes of an ultrasound pulse emitted immediately prior have decayed. The selection of the pulse repetition rate (PRF) to be used for this is explained in the following.

Taking into account the (Nyquist) sampling theorem (which, however, does not have to be considered here or, because only the relative velocity between the fluid and observation window has to be recorded, does not become satisfiable until the observation window is displaced), a maximum Doppler frequency of 59.53 kHz in a real-valued analysis would mean that a minimum pulse repetition rate or a minimum pulse repetition frequency of

PRFₘᵢₙ=2·df=119.06 kHz. (2)

would have to be set.

In the case of the implanted, vascular support systems in focus here, however, the following maximum pulse repetition rate PRFₘₐₓ results from the geometric consideration (maximum propagation distance of the ultrasound pulse) or the geometric boundary conditions in the support system and the resulting transit time of all relevant signal components:

$${PRF}_{max} = \frac{{c}_{Blood}}{d} = 27.93 kHz \qquad ( 3 )$$

Thus the maximum pulse repetition rate of the pulsed Doppler measurements here (or for the support systems in focus) is less than twice the maximum occurring Doppler shift.

These boundary conditions lead to a violation of the sampling theorem and consequently to an ambiguity of the measurement results, which can be remedied by an evaluation or method (displacing the observation window) as described in the following sections.

First, however, to illustrate the problems that occur with these boundary conditions, the resulting ambiguity is illustrated in FIGS. 4 and 5 (which can advantageously be avoided with the method presented here). FIG. 4 schematically shows an example Doppler frequency spectrum 40. FIG. 4 shows a Doppler shift at a volume flow of 3 l/min and a pulse repetition rate of approx. 25 kHz. The primary frequency component 41 (peak) is below the carrier frequency at approx. 0 Hz. FIG. 5 schematically shows a further example Doppler frequency spectrum 40. FIG. 5 shows a Doppler shift at a volume flow of 3 l/min and a pulse repetition rate of approx. 20 kHz. The primary frequency component 41 (peak) is at approx. +8 kHz. This illustrates in particular that different frequencies are output at different PRFs and the identical volume flow and, as a result, a volume flow set by the pump cannot be determined unambiguously without the use of the invention described here. At 20 kHz PRF, for example, the peak is at 3l/min at a frequency of approx. 8 KHz, which would in particular correspond to a velocity of 0.77 m/s or a volume flow of 0.9 l/min. However, the actual volume flow (to be measured) is 3 l/min in this example. These measurements were also carried out at an 8 MHz ultrasound frequency.

An example method in the sense of the solution proposed here, in which respective, ambiguous measurement results can advantageously be avoided, is described in the following sections.

For this purpose, it is proposed that the observation window be displaced at an observation window velocity, which is determined using an estimated flow velocity of the fluid (here the blood). This advantageously allows the Doppler shift to be transformed into a range that can be displayed without ambiguity using the selected ultrasound frequency and PRF. In connection with the displacement of the observation window, it is particularly advantageous if the radial cross-sections of the blood flow velocities are unchanged over a specific range (a few centimeters) in axial extension to the ultrasound element. The described method can be used in cardiac support systems of different types, for example in systems in aortic valve position as shown as an example in FIG. 1 or, for example, also in apically placed systems as shown as an example in FIG. 2.

An ultrasound-based pump volume flow measurement is usually based on one or more ultrasound transducers integrated into the support system and an optionally spatially offset (electronic) control and/or processing device, which can also be referred to as a measuring unit. The spatially offset control and/or processing device can be placed implanted and also placed extracorporeally connected by a transcutaneous lead. Together with the implantable vascular support system, it then forms a system for determining at least one flow parameter of the fluid flowing through the implantable vascular support system.

The described embodiments of FIG. 1 and FIG. 2 in particular require a pulsed Doppler measurement method (pulsed wave Doppler) in order to be able to position the observation window (the measurement region or the measurement window) along the main beam direction of the ultrasound transducer. The task of the control and/or processing device and/or the measuring unit is to produce suitable ultrasound pulses to be emitted by the ultrasound transducer or transducers, receive and amplify received scattered ultrasound energy (reflections, echo), and process the received signals to calculate a Doppler frequency spectrum.

Given the sufficiently known speed of sound in blood, the selection of the position of the observation window usually takes place via time intervals. After the emission of an ultrasound pulse, reflections from scatterers (e.g. blood cells) are immediately received directly in front of the transducer. Then, as the wavefront advances further, reflections from more distant regions are received. In a pulsed Doppler method, the received signals are processed only in a specific time interval temporally spaced apart from the time of emission of the ultrasound pulse.

The spatial distance of the observation window to the transducer plane of the ultrasound transducer can be selected or set via the selection of the time interval. The spatial extent of the observation window along the main beam direction of the ultrasound transducer can be selected or set via the length of the time interval.

A pulsed Doppler measurement usually consists of a large number of individual ultrasound pulses, i.e. a rapid sequence of emission and reception times with the frequency PRF (pulse repetition frequency). In this context, the PRF is in particular the duration from emission pulse to emission pulse. Changing the time interval between emission and measuring time interval from pulse to pulse, results in a moving observation window. In other words, this also means that, in order to displace the observation window, the time interval between an emission of an ultrasound pulse and a starting point of a measuring time interval has to be changed from ultrasound pulse to ultrasound pulse.

FIG. 6 schematically shows a detail view of a here proposed support system 10. The illustration of FIG. 6 relates to an example of a structure of a cardiac support system 10, in which a method proposed here can be used.

The ultrasound element 18 here represents the ultrasound sensor 18 and radiates in the direction of the blood flow velocity. In the region of an inlet cage 12 (provided with openings) of the support system 10, the inflowing blood 31 does not exhibit a constant flow profile yet. In the further course downstream in the regions 202 and 204, however, the radial flow profile is largely constant. The observation window 201 can thus advantageously be displaced in this region at the observation window velocity V_{Gate}. In the embodiments shown in FIG. 1 and FIG. 2, the regions 202 and 204 can be located in the channel 200, for example.

If, for example, as shown in the following equation (4), a flow velocity of v_{Blood}=3 m/s away from the piezo element of the ultrasound sensor 18 is to be measured in a fixed observation window at a PRF of 25 kHz and an ultrasound frequency of f₀=4 MHz, a Doppler shift of −15.58 kHz will result. At the given PRF of 25 kHz and the evaluation of positive and negative velocities, this Doppler shift can no longer be displayed in the negative part of the Doppler spectrum and is therefore displayed as 9.42 kHz in the positive frequency domain of the spectrum.

However, if (as proposed here) the observation window 201 is moved with a displacement velocity of v_{Gate}=1.75 m/s away from the piezo element of the ultrasound sensor 18, for example, the resulting (or relative) flow velocity to be transformed is reduced; here for example reduced to 3 m/s−1.75 m/s=1.25 m/s. At a PRF of 25 kHz, the resulting Doppler shift of −6.49 kHz can be displayed in the Doppler spectrum without ambiguity (see the following equation (7)).

$$\begin{matrix}{f}_{d , wrapped} = \frac{- 2 \cdot {v}_{Blood} \cdot {f}_{0}}{{c}_{0}} & ( 4 ) \\ \frac{- 2 \cdot 3 \frac{m}{s} \cdot 4 MHz}{1540 \frac{m}{s}} & ( 5 ) \\ = - 15 , 58 kHz & ( 6 ) \\ {f}_{d , trackingdoppl} = \frac{- 2 \cdot ( {v}_{Blood} - {v}_{Gate} ) \cdot {f}_{0}}{{c}_{0}} & ( 7 ) \\ = \frac{- 2 \cdot ( 3 \frac{m}{s} - 1 , 75 \frac{m}{s} ) \cdot 4 MHz}{1540 \frac{m}{s}} & ( 8 ) \\ = - 6 , 49 kHz & ( 9 )\end{matrix}$$

This is an example of how and that the observation window velocity can be determined such that a Doppler shift is transformed into a range that can be displayed without ambiguity.

A previously performed estimation of the flow velocity of the blood through the support system is in particular a basis for a corresponding determination of the observation window velocity here. This estimation is advantageously based on a previously performed ultrasound measurement (e.g. with a fixed observation window) using the ultrasound sensor 18 of the support system 10. However, this is only an example. The estimation could, for example, also be based on an empirical value, for example based on the age of the patient and/or the severity of the patient's disease.

FIG. 7 schematically shows example Doppler frequency spectra. The Doppler frequency spectra shown can, for example, result from the use of the method presented here.

FIG. 7 illustrates Doppler spectra at a blood flow velocity of 3 m/s at an ultrasound frequency of 4 MHz with an unfocused piezo element having a diameter of 6 mm and a PRF of 25 kHz. FIG. 7a illustrates the aliased (ambiguity-fraught) Doppler spectrum of a measurement with an observation window at a fixed distance of 25 mm from the piezo element. In contrast, FIG. 7b illustrates the non-aliased (unambiguous) Doppler spectrum with an observation window shifted 15 mm to 25 mm from the piezo element at a displacement velocity (observation window velocity) of 1.75 m/s.

Two deflections or peaks can furthermore be seen in each of the Doppler spectra shown in FIG. 7, namely a peak resulting from the Doppler shift caused by the aortic wall (non-moving scatterer) 42 and a peak caused by reflection on moving scatterers (e.g., blood cells) 43. In FIGS. 7 and 8, the solid lines describe results of a Fourier transformation and the dashed lines describe results of the so-called Welch method.

FIG. 7 illustrates how aliasing can be prevented by the method described here. FIG. 7b further shows how displacing the observation window on the right side of the spectrum results in a second peak 42 beyond 0 Hz. This peak 42 (which describes the Doppler shift of the non-moving scatterer aortic wall, for example) results from the relative movement of the observation window to the stationary tissue, e.g. the aortic wall, and thus shows the Doppler frequency of the observation window or the Doppler frequency which affects the observation window velocity.

FIG. 7b also shows that the peak widths of the two peaks (in comparison to the peak widths in FIG. 7a) change due to the movement of the observation window. Peak 42, which is caused by reflection on the stationary tissue of the aortic wall, widens. In contrast, peak 43, which is caused by scatterers (such as blood cells) moving at the blood flow velocity, narrows.

FIG. 8 schematically shows other example Doppler frequency spectra. The Doppler frequency spectra shown can, for example, result from the use of the method presented here.

The deterioration of the signal-to-noise ratio (SNR), which can be seen in FIG. 7, is a consequence of a mismatch between the observation window velocity and the sampling rate of the received signal, which causes the observation window to jitter. Reducing the jitter, and thereby improving the SNR in the spectrum, can, for example, be achieved by adapting the sampling frequency to the observation window velocity, resampling the received signal and/or oversampling.

The following equation (10) shows how the observation window velocity of the observation window and the sampling rate can be adapted to one another in a particularly advantageous manner. FIG. 8 illustrates the aforementioned possibilities for improving the SNR.

Equation 10 shows how the velocity of the observation window can be selected in a particularly advantageous manner in order to maximize the SNR at the given speed of sound in blood c₀, a given PRF and a given sampling rate fₛ.

$${v}_{Gate} = n \cdot \frac{PRF \cdot {c}_{0}}{2 \cdot {f}_{S}} n ∈ Z \qquad ( 10 )$$

This is an example that, and, if applicable, of how, the observation window velocity and a sampling rate can be adapted to one another.

FIGS. 8a, 8b and 8c respectively show a Doppler spectrum, after the use of the method described here, at a flow velocity of 3 m/s, when using a non-focused piezo element having a diameter of 4 mm, an ultrasound frequency of 8 MHz and a PRF of 40 kHz. In each of the measurements, the result of which is illustrated in FIGS. 8a, 8b and 8c, the observation window moves from a distance of 15 mm to a distance of 30 mm from the piezo element.

In FIGS. 8a and 8b, the observation window moved at a (observation window) velocity of 1.75 m/s. A sampling rate of 20 MHz was used for FIG. 8a, and a sampling rate of 100 MHz was used for FIG. 8b. FIG. 8c illustrates the SNR with an adapted sampling rate of 20 MHz and a displacement velocity of the observation window of 1.54 m/s.

When using the method described here, it is advantageously possible to achieve another goal, namely the reduction of the spectral widening of the sought frequency peak at high blood flow velocities. This additional effect can usually not be achieved when using evaluation methods (with a fixed observation window) that do not work according to the method described here. Based on these narrower frequency peaks in the Doppler spectrum caused by the flow velocity of the blood, the accuracy of the determination (estimation) of the primary velocity component can be improved significantly.

Displacing the observation window at v_{Gate}, the roughly estimated flow velocity of the moving scatterers (such as blood cells) in the blood, prolongs the dwell time in the observation window for all moving scatterers for which |v_{Blood}−v_{Gatel}|<v_{Blood}. This can advantageously lead to an improvement of the SNR (amplitude) of \sqrt{(N)} due to the integration gain in the subsequent Fourier transformation, whereby N corresponds to the number of samples while the scatterer is in the observation window.

For the static scatterers, e.g. the aortic wall, for which the condition |v_{Blood}−v_{Gate}|<v_{Blood} is not fulfilled, the scatterers no longer move in the observation window during the entire observation period as in a conventional evaluation. By using the method described here, this duration is shortened significantly, in particular as a function of the flow velocity of the blood or the velocity of the observation window. This can also be described in other words as follows: In the case of a stationary window and “one” stationary scatterer, the entire wave train is reflected on it.

Consequently, if the observation duration is selected to be less than/equal to the pulse duration of the wave train, a portion of the pulse is reflected on it during the entire observation period. This long dwell time in the observation window (time domain) produces a narrow-band peak in the spectrum (frequency domain). Moving the window shortens the dwell time, and the peak in the spectrum becomes more broad-banded. As shown in FIG. 7, the resulting reduction of the integration gain (in comparison to known methods) leads to a widening of the frequency peak caused by static scatterers (which is now no longer at 0 Hz) and to a smearing of the signal energy in the spectrum.

An additional special advantage of the method described here is that the displacement velocity of the observation window (observation window velocity) can be freely selected within certain limits. If, for example, the static scatterers observed at v_{Gate}, which experience a Doppler shift of

$${f}_{d , start} = \frac{2 \cdot {v}_{Gate} \cdot {f}_{0}}{{c}_{0}} \qquad ( 11 )$$

are in the same frequency domain as the sought Doppler shift caused by the blood flow (not two, but only one peak is detected in the spectrum), the displacement velocity of the observation window can advantageously be changed slightly, so that the sought frequency peak is no longer covered by the significantly stronger frequency peak caused by static scatterers. This effect is shown schematically in FIG. 9. The reduction of the spectral widening is not taken into account in this schematic illustration.

FIG. 9 schematically shows other example Doppler frequency spectra. The Doppler frequency spectra shown can, for example, result from the use of the method presented here.

By slightly changing the displacement velocity v_{Gate} of the observation window, the covering of the sought frequency peak caused by the blood flow velocity by the frequency peak caused by the static scatterers can be eliminated.

In FIG. 9a, the sought Doppler frequency of the flow velocity 44 is covered. In FIG. 9b, the sought Doppler frequency of the flow velocity 44 is no longer covered. The displacement velocity of the observation window (observation window velocity) was changed slightly to do this. This is an example that, and, if applicable, of how, the observation window velocity can be determined such that the measurement result of the pulsed Doppler measurement and a Doppler shift caused by static scatterers are spaced apart from one another.

The solution presented here in particular enables one or more of the following advantages:
PWD-based flow velocity or volume flow measurement is possible even with a large distance between the measurement window and the ultrasound transducer.
Resolution of the geometrically caused ambiguity of the Doppler shift due to geometric boundary conditions in the support system (VAD).
Reduction of the spectral widening.
Increase in the accuracy of the Doppler frequency estimation.
More accurate determination of the flow velocity.
Preventing the sought Doppler frequency shift from being covered by the frequency peak in the Doppler spectrum caused by static scatterers, e.g. the aortic wall.

The system 45 shown in FIG. 10 comprises an implantable vascular support system 10 and includes a control and/or processing device 46 for determining at least one flow parameter of a fluid 31 flowing through the implantable vascular support system 10. The control and/or processing device 46 is connected to the implantable vascular support system 10 by a transcutaneous lead and can be placed extracorporeally. It should be noted that the control and/or processing device 46 can in principle also be designed to be implanted in the human body.

In the control and/or processing device 46, there is a device 48 for estimating the flow velocity of the fluid 31. The control and processing device 46 comprises a device 50 for carrying out a pulsed Doppler measurement using an ultrasound sensor 18 shown in FIG. 6 in an observation window 201 shown in FIG. 6 inside the support system 10, whereby the observation window 201 is displaced at an observation window velocity which is determined using the estimated flow velocity.

The device 50 for carrying out a pulsed Doppler measurement is designed to displace the observation window 201 by changing the time interval between an emission of an ultrasound pulse and a measuring time interval from ultrasound pulse to ultrasound pulse.

The control and processing device 46 further comprises a device 52 for determining the at least one flow parameter of the fluid using at least one measurement result of the pulsed Doppler measurement or a measurement result of the pulsed Doppler measurement and the observation window velocity. The device 52 for determining the at least one flow parameter of the fluid is designed to determine a flow velocity of the fluid using a measurement result of the pulsed Doppler measurement and the observation window velocity by adding together the observation window velocity and a relative velocity determined on the basis of the pulsed Doppler measurement.

The device 48 for estimating the flow velocity of the fluid 31 is used to estimate the flow velocity of the fluid 31 on the basis of an operating parameter of a flow machine of the support system 10.

The observation window velocity of the observation window 201 of the device for carrying out a pulsed Doppler measurement is designed to transform a Doppler shift into a range that can be displayed without ambiguity, whereby the observation window velocity corresponds substantially to a flow velocity estimated in the device 48 for estimating the flow velocity of the fluid 31.

In the system, the observation window velocity and a sampling rate of the fluid 31 flowing through the implanted vascular support system 10 are adapted to one another. The observation window velocity is determined such that the measurement result of the pulsed Doppler measurement and a Doppler shift caused by static scatterers are spaced apart from one another.

### LIST OF REFERENCE SKINS

10 Support system
12 Inlet cage
18 Ultrasound sensor
19 Bypass
20 Heart
21 Left ventricle
22 Aorta
23 Aortic valve
31 Fluid volume flow/blood flow
32 Total fluid volume flow
40 Doppler frequency spectrum
41 Primary frequency component
42 Peak due to Doppler shift
43 Peak due to moving scatterers
44 Flow velocity
45 System
46 Control and/or processing device
48 Device for estimating the flow velocity
50 Device for carrying out a pulsed Doppler measurement
52 Device for determining a flow parameter
200 Channel
201 Observation window

1-19. (canceled)

20. A method for determining at least one flow parameter of blood in a cardiac support system, the method comprising:
estimating a flow velocity of the blood;
performing a pulsed Doppler measurement using an ultrasound sensor of the cardiac support system to generate at least one Doppler measurement result,
wherein, during the pulsed Doppler measurement, the ultrasonic sensor is within an observation window within a cannula-like section of the cardiac support system, and
wherein the observation window is displaced at an observation window velocity that is based on the estimated flow velocity;
determining the at least one flow parameter of the blood based on at least one Doppler measurement result.

21. The method of claim 20, wherein determining the at least one flow parameter of the blood comprises determining the flow velocity of the blood based on the at least one Doppler measurement result and the observation window velocity.

22. The method of claim 20, wherein the flow velocity is estimated based on an operating parameter of a flow machine of the cardiac support system.

23. The method of claim 20, further comprising determining the observation window velocity so that a Doppler shift is transformed into a range that can be displayed without ambiguity on a frequency spectrum.

24. The method of claim 20, wherein the observation window velocity corresponds substantially to the estimated flow velocity.

25. The method of claim 20, further comprising displacing the observation window by changing a time interval between an emission of an ultrasonic pulse and a start time of a measurement time interval between ultrasonic pulses.

26. The method of claim 20, wherein the observation window velocity and a sampling rate are correlated with one another.

27. The method of claim 20, further comprising determining the observation window velocity such that the at least one Doppler measurement result and a Doppler shift caused by static scatterers are spaced apart from one another on a frequency spectrum.

28. The method of claim 20, wherein determining the at least one flow parameter of the blood comprises determining the flow velocity of blood by adding together the observation window velocity and a relative velocity determined based on the at least one Doppler measurement result.

29. A cardiac support system, the cardiac support system comprising:
a controller configured to determine at least one flow parameter of blood flowing through the cardiac support system, the controller comprising:
a device configured to estimate a flow velocity of the blood,
a device configured to carry out a pulsed Doppler measurement using an ultrasonic sensor to generate at least one Doppler measurement result,
wherein, during the pulsed Doppler measurement, the ultrasonic sensor is within an observation window within a cannula-like section of the cardiac support system, and
wherein the observation window is displaced at an observation window velocity that is based on the estimated flow velocity; and
a device configured to determine at least one flow parameter of the blood based on at least one Doppler measurement result.

30. The system of claim 29, wherein the device configured to determine the at least one flow parameter of the blood is configured to determine a flow velocity of the blood based on at least one Doppler measurement result and the observation window velocity.

31. The system of claim 29, wherein the device configured to estimate the flow velocity of the blood is configured to estimate the flow velocity of the blood based on an operating parameter of a flow machine of the cardiac support system.

32. The system of claim 29, wherein the observation window velocity of the observation window is selected so that a Doppler shift is in a range that can be displayed without ambiguity on a frequency spectrum.

33. The system of claim 29, wherein the observation window velocity corresponds substantially to the estimated flow velocity.

34. The system of claim 29, wherein the device configured to carry out a pulsed Doppler measurement is configured to displace the observation window by changing the time interval between an emission of an ultrasound pulse and a start time of a measurement time interval from ultrasound pulse to ultrasound pulse.

35. The system of claim 29, wherein the observation window velocity and a sampling rate of the blood flowing through the cardiac support system are correlated with one another.

36. The system of claim 29, wherein the observation window velocity is determined such that the measurement result of the pulsed Doppler measurement and a Doppler shift caused by static scatterers are spaced apart from one another on a frequency spectrum.

37. The system of claim 29, wherein the device configured to determine the at least one flow parameter of the blood is configured to determine a flow velocity of the blood by adding together the observation window velocity and a relative velocity determined on the basis of the pulsed Doppler measurement.
